(19) United States
(12) Patent Application Publication
(10) Pub. No.: US 2014/0283155 A1
(43) Pub. Date: Sep. 18, 2014

(54) REPRESSIBLE LETHAL SYSTEM FOR REGULATING INSECT POPULATIONS

(71) Applicant: California Institute of Technology, Pasadena, CA (US)

(72) Inventors: Omar S. Akbari, Pasadena, CA (US); Katie L. Kennedy, Los Angeles, CA (US); Bruce A. Hay, Encino, CA (US)

(21) Appl. No.: 14/206,011

(22) Filed: Mar. 12, 2014

Related U.S. Application Data
(60) Provisional application No. 61/779,730, filed on Mar. 13, 2013.

(51) Int. Cl. A01K 67/033

(52) U.S. Cl. CPC A01K 67/0333
USPC 800/13; 435/320.1; 800/21

(57) ABSTRACT

Disclosed herein are components, systems and methods for regulating population of multicellular organisms, for example insects.

### RELATED APPLICATIONS

The present application claims priority under 35 U.S.C. §119(e) to U.S. Provisional Application Nos. 61/779,730, filed Mar. 13, 2013, which is herein expressly incorporated by reference in its entirety.

### REFERENCE TO SEQUENCE LISTING

The present application is being filed along with a Sequence Listing in electronic format. The Sequence Listing is provided as a file entitled SEQLISTING_CALTE.099A.TXT, created Mar. 11, 2014, which is 18.8 Kb in size. The information in the electronic format of the Sequence Listing is incorporated herein by reference in its entirety.

### STATEMENT REGARDING FEDERALLY SPONSORED R&D

This invention was made with government support under DP1OD003878 awarded by the National Institutes of Health. The government has certain rights in the invention.

### BACKGROUND

1. Field of the Invention

The present application relates generally to components, methods and systems that can be used for regulating populations of multicellular organisms, for example insects.

2. Description of the Related Art

Establishing effective control measures for biological control, for example controls for wild insect populations, is a complex, continuing and difficult problem, and new technologies need to be developed to stop the spread of vector borne diseases and the destruction of agricultural crops. Some existing methods for controlling insect populations utilize genetically modified crops expressing insect toxins, non-species specific pesticides, or the introduction and release of natural predators. While these methods are generally effective, there is a constant appearance of insects that evolve resistance to these approaches, resulting in the need for new technologies. Additionally, blanketing crops with insecticides is not cheap or environmentally friendly, and genetically modifying every single crop species is not socially accepted, cannot solve all pest related problems, and remains to be implemented for most crops. Furthermore, while the release of natural predators can be effective, for most insects, no potent natural predator can be easily mass reared and released.

Sterile insect technique (SIT) has been shown to be a species specific and environmentally friendly method for insect population control. However, SIT is critically dependent on males having high fitness, which is often comprised by treatments such as ionizing radiation or chemicals that are used to induce sterility and requires sex-separation that is difficult to handle for many insects. A modified SIT technology known as release of insects carrying a dominant lethal (RIDL) has been recently developed (Thomas et al., Science 287, 2474-2476, 2000). This RIDL system involves the expression of a tetracycline repressible transactivator fusion protein (tTa), which binds to the tetracycline-responsive element (tRe), driving expression of a toxin in the absence of the tetracycline. In the presence of tetracycline, the RIDL system is silenced, and since the toxin is not produced, progenies survive. Thus, to mass rear insects carrying the RIDL system, a diet supplemented with large amounts of tetracycline need to be provided to progenies. However, growth on tetracycline has numerous unwanted side effects on insects, such as loss of the gut microbiome, loss of symbiotic bacteria, and effects on mitochondrial function. In addition, the feeding of tetracycline has recently been shown to impose a large fitness cost in insects (Zeh et al., Sci. Rep. 2:375 (2012)).

Accordingly, there is still a need for an effective, safe and environmentally friendly method for biological control of organisms, such as insects.

### SUMMARY

Some embodiments provide a recombinant insect. The recombinant insect, in some embodiments, comprises a lethal genetic element that includes: a first polynucleotide encoding one or more toxins, wherein the first polynucleotide is operably linked with a first promoter; and a binding sequence for a repressible transactivator protein, wherein the binding sequence is operably linked with the first promoter and expression of the one or more toxins is activatable by the repressible transactivator protein; wherein the binding of the repressible transactivator protein to the binding sequence is repressible by vanillic acid, analogs or derivatives thereof.

In some embodiments, the recombinant insect further comprises a regulatory genetic element that comprises a second polynucleotide encoding the repressible transactivator protein, wherein the second polynucleotide is operably linked with a second promoter. In some embodiments, the lethal genetic element and the regulatory element are located on the same chromosome of the recombinant insect. In some embodiments, the lethal genetic element and the regulatory element are located on different chromosomes of the recombinant insect

In some embodiments, the repressible transactivator protein is VanR. In some embodiments, the binding sequence for the repressible transactivator protein is VanO operator sequence. In some embodiments, at least one of the one or more toxins is a cell death protein, a restriction endonuclease, or a microRNA. In some embodiments, the first promoter is a P_hsp70min promoter, a CMVmin promoter, a drosophila minimal P-element promoter, or any variant thereof. In some embodiments, the first promoter is a female specific promoter. In some embodiments, the second promoter is a ubiquitous promoter. In some embodiments, the ubiquitous promoter is selected from the group consisting of baculovirus immediate early promoter. In some embodiments, the first polynucleotide encoding one or more toxins, the second polynucleotide encoding the repressible transactivator protein, or both of the first polynucleotide and the second polynucleotide comprise a female specific intron.

Some embodiments provide a vector, where the vector comprises a lethal genetic element that comprises: a first polynucleotide encoding one or more toxins, wherein the first polynucleotide is operably linked with a first promoter; and a binding sequence for a repressible transactivator protein, wherein the binding sequence is operably linked with the first promoter and expression of the one or more toxins is activatable by the repressible transactivator protein; wherein the binding of the repressible transactivator protein to the binding sequence is repressible by vanillic acid, analogs or derivatives thereof

In some embodiments, the vector further comprises a regulatory genetic element that comprises a second polynucleotide encoding the repressible transactivator protein, wherein the second polynucleotide is operably linked with a second promoter. In some embodiments, the lethal genetic element and the regulatory genetic element are configured to allow autonomous expression of the one or more toxins and the repressible transactivator protein. In some embodiments, the lethal genetic element and the regulatory genetic element are separated by one or more insulators.

Some embodiments provide a method for regulating insect population, where the method includes: providing a stock of recombinant insects, wherein at least one of the recombinant insect comprises a lethal genetic element, wherein the lethal genetic element comprises: a first polynucleotide encoding one or more toxins, wherein the first polynucleotide is operably linked with a first promoter; and a binding sequence for a repressible transactivator protein, wherein the binding sequence is operably linked with the first promoter and expression of the one or more toxins is activatable by the repressible transactivator protein; wherein the binding of the repressible transactivator protein to the binding sequence is repressible by vanillic acid, analogs or derivatives thereof; and distributing the stock of recombinant insects into an environment lacking vanillic acid, analogs or derivatives thereof, whereby individual recombinant insects breed with insects in the environment to produce offspring expressing the one or more toxins. In some embodiments, the first polynucleotide further comprises a coding region for the repressible transactivator protein, wherein a 2A sequence is located between the coding region for the repressible transactivator protein and the coding region of the one or more toxins.

In some embodiments, the method comprises breeding the stock of recombinant insects in the presence of vanillic acid, analogs or derivatives thereof before distributing the stock of recombinant insects into the environment. In some embodiments, the expression of the one or more toxins is sex-specific. In some embodiments, the expression of the one or more toxins is controlled by a sex-specific promoter, a sex-specific enhancer, or by sex-specific splicing. In some embodiments, only male recombinant insects are distributed to the environment.

Some embodiments provide a recombinant insect, wherein the recombinant insect comprises a lethal genetic element that comprises: a polynucleotide comprising a first coding region for a toxin, a second coding region for a repressible transactivator protein, and a 2A sequence located between the first coding region and the second coding region, wherein the polynucleotide is operably linked with a promoter; and a binding sequence for the repressible transactivator protein, wherein the binding sequence is operably linked with the promoter and expression of the toxin is activatable by the repressible transactivator protein; wherein the binding of the repressible transactivator protein to the binding sequence is repressible by vanillic acid, analogs or derivatives thereof. In some embodiments, the first coding region, the second coding region, or both the first and the second coding region comprise a female specific intron. In some embodiments, the second coding region is located upstream of the first coding region.

### BRIEF DESCRIPTION OF THE DRAWINGS

FIG. 1A is a schematic illustration showing that the behavior of insects carrying a VanDal repressible lethal system described herein in the presence or absence of vanillic acid in their diet. In the presence of vanillic acid, the insects live, while the insects die in the absence of vanillic acid.

FIG. 1B is a schematic illustration of a non-limiting example of the VanDal repressible lethal system described herein.

FIG. 1C is a schematic illustration to show how a VanDal repressible lethal system described herein, in some embodiments, functions to lead to the death of insects in the absence of vanillic acid.

FIG. 1D is a schematic illustration to show how the VanDal repressible lethal system described herein, in some embodiments, functions to maintain the live of insects in the presence of vanillic acid,

FIGS. 2A-D are schematic illustrations showing non-limiting examples of female-specific VanDal repressible lethal system described herein. “Fs-intron” represents female specific intron, and “Fs Promoter” represents female specific promoter.

FIG. 3A-D are schematic illustrations showing non-limiting examples of the feedback VanDal repressible lethal system described herein. “Fs-intron” represents female specific intron.

### DETAILED DESCRIPTION

In the following detailed description, reference is made to the accompanying drawings, which form a part hereof. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

The present application provides components, systems and methods for regulating populations of multicellular organisms (e.g., insects). For example, the components, systems and methods disclosed herein can be used to control, suppress or eliminate one or more wild insect populations. In some embodiments, the method disclosed herein relies on the presence or absence of vanillic acid, analogs or derivatives thereof in an environment to control the fate (that is, live or death) of a multicellular organism (for example, an insect) in the environment. In some embodiments, the method includes distributing one or more recombinant insects to the environment, wherein the one or more recombinant insects comprises a lethal genetic element that comprises: a first polynucleotide encoding one or more toxins, wherein the first polynucleotide is operably linked with a first promoter; and a binding sequence for a repressible transactivator protein, wherein the binding sequence is operably linked with the first promoter and expression of the one or more toxins is activatable by the repressible transactivator protein; and wherein the binding of the repressible transactivator protein to the binding sequence is repressible by vanillic acid, analogs or derivatives thereof. Also disclosed are Vanillic acid suppressible dominant lethal (VanDal) systems that relies on presence or absence of vanillic acid, analogs or derivatives thereof in an environment to control the fate of a multicellular organism (for example, an insect) in the environment.

### Definitions

Unless defined otherwise, technical and scientific terms used herein have the same meaning as commonly understood by one of ordinary skill in the art to which this invention belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present invention, the following terms are defined below.

As used herein, the term “vector” refers to a polynucleotide construct, typically a plasmid or a virus, used to transmit genetic material to a host cell. Vectors can be, for example, viruses, plasmids, cosmids, or phage. A vector as used herein can be composed of either DNA or RNA. In some embodiments, a vector is composed of DNA. An “expression vector” is a vector that is capable of directing the expression of a protein encoded by one or more genes carried by the vector when it is present in the appropriate environment. Vectors are preferably capable of autonomous replication. Typically, an expression vector comprises a transcription promoter, a gene, and a transcription terminator. Gene expression is usually placed under the control of a promoter, and a gene is said to be “operably linked to” the promoter.

As used herein, the term “operably linked” is used to describe the connection between regulatory elements and a gene or its coding region, and the connection between a regulatory element (for example, an operator, a transcription factor binding sequence, or a promoter) and another regulator element. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence. As another example, a transcription factor binding sequence is operably linked to a promoter if the transcription factor binding sequence functions as a regulator (e.g., acts as an on/off switch) for the transcription driven by the promoter.

The term “construct,” as used herein, refers to a recombinant nucleic acid that has been generated for the purpose of the expression of a specific nucleotide sequence(s), or that is to be used in the construction of other recombinant nucleotide sequences.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The term “regulatory element” and “expression control element” are used interchangeably and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and a ribosome binding sites. Regulatory elements that are used in eukaryotic cells can include, without limitation, transcriptional and translational control sequences, such as promoters, enhancers, insulators, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, 2A sequences or elements refer to small peptides introduced as a linker between two proteins, allowing autonomous intraribosomal self-processing of polyproteins (See e.g., de Felipe. Genetic Vaccines and Ther. 2:13 (2004); deFelipe et al. Traffic 5:616-626 (2004)). These short peptides allow co-expression of multiple proteins from a single vector. Many 2A sequences are known in the art. Examples of 2A sequences that can be used in the methods, system and components disclosed herein, without limitation, include 2A sequences from the foot-and-mouth disease virus (F2A), equine rhinitis A virus (E2A), Thosea asigna virus (T2A), and porcine teschovirus-1 (P2A) (e.g., as described in U.S. Patent Publication No. 20070116690.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “enhancer” refers to a type of regulatory element that can increase the efficiency of transcription, regardless of the distance or orientation of the enhancer relative to the start site of transcription.

As used herein, the term “insulator” refers to a genetic boundary element that can block the interaction between enhancers and promoters, so that expression of a gene (including induction or repression of the expression of the gene) does not interfere with that of the neighboring gene.

As used herein, the term “variant” refers to a polynucleotide (or polypeptide) having a sequence substantially similar to a reference polynucleotide (or polypeptide). In the case of a polynucleotide, a variant can have deletions, substitutions, and/or additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, and/or additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polypeptide as determined by sequence alignment programs known by skilled artisans.

As used herein, the term “transformation” refers to the introduction of a nucleic acid (for example a plasmid) into a host or its progenies. For example, the nucleic acid can be introduced to an insect or its progenies by injecting with a recombinant vector comprising the lethal genetic element and/or the regulatory genetic element as described below to the germline of the insect.

### Vectors Comprising Lethal Gnetic Elements

Disclosed herein are vectors, such as plasmids, comprising lethal genetic elements regulable by vanillic acid, analogs or derivatives thereof. In some embodiments, the lethal genetic element comprises: a polynucleotide encoding one or more toxins, wherein the polynucleotide is operably linked with a first promoter; and a binding sequence for a repressible transactivator protein, wherein the binding sequence is operably linked with the first promoter and expression of the one or more toxins is activatable by the repressible transactivator protein; wherein the binding of the repressible transactivator protein to the binding sequence is repressible by vanillic acid, analogs or derivatives thereof. The first promoter can, in some embodiments, drive the expression of the one or more toxins in a host organism (e.g. a host insect). Vanillic acid (4-hydroxy-3-methoxybenzoic acid) is a non-toxic dihydroxybenzoic acid derivative that has been used as a flavoring agent. In some embodiments, the vector further comprises a regulatory genetic element that comprises a polynucleotide encoding the repressible transactivator protein, wherein the polynucleotide is operably linked with a second promoter. The second promoter can, in some embodiments, drive the expression of the repressible transactivator protein in a host organism (e.g. a host insect).

The relative location of the lethal genetic element and the regulatory genetic element on the vector is not particularly limited. For example, the lethal genetic element and the regulatory genetic element can be adjacent to each other on the vector. In some embodiments, the lethal genetic element and the regulatory genetic element are configured to allow autonomous expression of the one or more toxins and the repressible transactivator protein. For example, the transcription direction driven by the first promoter of the lethal genetic element can be in the opposite direction of the transcription direction driven by the second promoter of the regulatory element on the vector. As another example, one or more insulators can be present between the lethal genetic element and the regulatory genetic element to allow autonomous expression of the one or more toxins and the repressible transactivator protein.

The lethal genetic element and regulatory genetic element can, in some embodiments, be located in two different vectors. In these embodiments, the two different vectors can be co-transformed into an organism, such as an insect, for the lethal genetic element and the regulatory genetic element to work in concert to achieve regulation of biological control.

In some embodiments, the polynucleotide encoding one or more toxins comprises a coding region for a repressible transactivator protein. In some embodiments, a linker sequence that allows self-processing of polypeptides is located between the coding region for the one or more toxins and the coding region for the repressible transactivator protein. The linker sequence can, in some embodiments, be a 2A sequences including but are not limited to 2A sequences from the foot-and-mouth disease virus (F2A), equine rhinitis A virus (E2A), Thosea asigna virus (T2A), and porcine teschovirus-1 (P2A). The expression of the one or more toxins and the repressible transactivator in the lethal genetic element can be sex non-specific or sex-specific. For example, the coding region for the one or more toxins can comprise a female specific intron. As another example, the coding region for the repressible transactivator protein can comprise a female specific intron. In still another example, both of the coding region for the one or more toxins and the coding region for the repressible transactivator protein comprise a female specific intron. The relative location of the coding region for the one or more toxins and the coding region for the repressible transactivator protein can vary. For example, the coding region for the one or more toxins can be located upstream or downstream of the coding region for the repressible transactivator protein. In some embodiments, the coding region for the one or more toxins is located upstream of the coding region for the repressible transactivator protein.

In some embodiments, the vectors disclosed herein can further contain genetic elements that allow the integration of the lethal genetic element and/or the regulator genetic element into the chromosome of an organism (for example a multicellular organism including insects). The integration site on the chromosome of the organism can be random or predetermined. For example, either or both of the lethal genetic element and the regulatory genetic element can be present in a piggyback transposable element to allow the integration of the lethal genetic element and/or the regulatory genetic element into the chromosome of the organism which the vector is transformed into. Without being bound by any particular theory, it is believed that the piggyback transposable element carrying either or both of the lethal genetic element and the regulatory element can randomly integrate into the genome of an organism (e.g., an insect chromosome). In some embodiments, a site-specific recombination system such as FRT/FLP and CRE/LOX recombination system can be used to insert either or both of the lethal genetic element and regulatory genetic element into the chromosome of a target organism through homologous recombination. In some embodiments, the integration site is predetermined.

The vectors described herein can include prokaryotic replicons (that is, DNA sequences having the ability to direct autonomous replication and maintenance of the recombinant DNA molecule extrachromosomally in a prokaryotic host cell), such as a bacterial host cell, transformed therewith. Such replicons are well known in the art. In addition, vectors that include prokaryotic replicons may also include genes whose expression confers a detectable marker such as a drug resistance. Typical bacterial drug resistance genes are those that confer resistance to ampicillin, kanamycin, and tetracycline.

In some embodiments, the vector can include one or more genes for selectable markers that are effective in a eukaryotic cell, such as a drug resistance selection marker, a fluorescent protein, or a protein that can result in visible phenotypic change of the transformed organism (e.g., change in eye color or hair color). In the case of a fluorescent protein marker (e.g., GFP) expressed under the control of a suitable promoter, this protein can be visualized simply by illuminating with a suitable excitatory wavelength (e.g. blue) and observing the fluorescence. Such a marker would also allow easy identification of trapped insects in release-and-recapture experiments. Other suitable markers for transformation are known in the art, and can be chosen by one of skilled in the art according to the desired use.

Generation of the vectors described herein can be accomplished using any suitable genetic engineering techniques well known in the art, including, without limitation, the standard techniques of restriction endonuclease digestion, ligation, transformation, plasmid purification, and DNA sequencing, for example as described in Sambrook et al. (Molecular Cloning: A Laboratory Manual. Cold Spring Harbor Laboratory Press, N.Y. (1989)). The vectors can incorporate sequences from the genome of any known organism. The sequences can be incorporated in their native form or can be modified in any way to obtain a desired activity. For example, the sequences can comprise insertions, deletions or substitutions.

Various promoters can be operably linked with the polynucleotide encoding one or more toxins. In some embodiments, the promoter can drive the expression of at least one of the one or more toxins in an organism (e.g., an insect) transformed with the vector. The promoter can be naturally-occurring or non-naturally occurring.

Examples of promoters that are operably linked with the polynucleotide encoding one or more toxins in the lethal genetic element, include, but are not limited to, viral promoters, plant promoters and mammalian promoters. Non-limiting examples of the promoters that can be used to be operably linked with the polynucleotide encoding one or more toxins include heat shock promoters (e.g., a Hsp70 promoter (including P_Hsp70^{min} promoter), cytomegalovirus (CMV) immediate early promoter (including human CMV^{min} promoter), drosophila minimal P-element promoter (Pmin), CAG promoter (which is a combination of the CMV early enhancer element and chicken beta-actin promoter, described in Alexopoulou et al. BMC Cell Biology 9:2, (2008)), simian virus 40 (SV40) promoter, the 35S RNA and 19S RNA promoters of cauliflower mosaic virus (CaMV) described in Brisson et al., Nature 1984, 310:511-514, the coat protein promoter to tobacco mosaic virus (TMV), heat shock promoters, such as soybean hsp17.5-E or hsp17.3-B described in Gurley et al., Mol. Cell. Biol. 1986, 6:559-565, and any variants thereof.

In some embodiments, the promoter is a P_Hsp70^{min} promoter. In some embodiments, the promoter comprise a nucleic acid sequence having at least about 90%, at least about 95%, at least about 96%, at least about 97%, at least about 98%, at least about 99%, or more, sequence identity to SEQ ID NO: 7.

In some embodiments, the repressible transactivator protein comprises a portion or entire sequence of transcription regulator VanR, or any variants thereof. In some embodiments, the repressible transactivator protein is transcription regulator VanR or variants thereof. For example, the vanillic acid repressible transactivator protein can be Caulobacter Crescentus VanR protein or variants thereof. VanR proteins or homologs thereof from prokaryotes that use vanillic acid as a carbon source can also be used as the transcription regulator VanR in the methods, components and systems described herein. Non-limiting examples of the repressible transactivator protein include GntR family transcriptional regulators from Sphingomonas sp. ATCC 31555, Marinomonas sp. D104, Pseudomonas chlororaphis, Pseudomonas putida H8234, YP_{—}008095667, Sphingomonas elodea (for example, the proteins of GenBank accession nos. WP_{—}019369237, WP_{—}024025167, WP_{—}023969037, YP_{—}008095667, WP_{—}010545285), VanR from Pseudomonas protegens Pf-5 (e.g., GenBank accession no. YP_{—}260576) and Pseudomonas nitroreducens (for example, the proteins of GenBank accession no. ACP17983), and variants thereof. In some embodiments, the polynucleotide encoding the repressible transactivator protein is a nucleic acid with the sequence of SEQ ID NO: 2. In some embodiments, the polynucleotide encoding the repressible transactivator protein comprises a nucleic acid with the sequence of SEQ ID NO: 2. In some embodiments, the polynucleotide encoding the repressible transactivator protein comprise a nucleic acid sequence having at least about 75%, at least about 85%, at least about 80%, at least about 85%, at least about 90%, at least about 95%, at least about 96%, at least about 97%, at least about 98%, at least about 99%, or more, sequence identity to SEQ ID NO: 2. In some embodiments, the repressible transactivator protein is a protein with the sequence of SEQ ID NO: 10. In some embodiments, the repressible transactivator protein is a protein comprising the sequence of SEQ ID NO: 10. In some embodiments, the repressible transactivator protein comprises an amino acid sequence having at least about 90%, at least about 95%, at least about 96%, at least about 97%, at least about 98%, at least about 99%, or more, sequence identity to SEQ ID NO: 10. In some embodiments, the repressible transactivator protein is a fusion protein comprising transcription regulator VanR. For example, one or more transcription domains can be fused to the N-terminus and/or C-terminus of the VanR protein to create a highly efficient repressible transactivator. In some embodiments, the N-terminus or the C-terminus of the VanR is fused to VP16 or the acidic domain of VP16. In some embodiments, the N-terminus or the C-terminus of the VanR is fused to Ga14 transactivation domain. In some embodiments, the repressible transactivator protein is a fusion protein formed by VanR and VP16. For example, the polynucleotide encoding the repressible transactivator protein can be a nucleic acid with the sequence of SEQ ID NO: 3. In some embodiments, the polynucleotide encoding the repressible transactivator protein is a nucleic acid comprising the sequence of SEQ ID NO: 3. In some embodiments, the polynucleotide encoding the repressible transactivator protein comprise a nucleic acid sequence having at least about 75%, at least about 85%, at least about 80%, at least about 85%, at least about 90%, at least about 95%, at least about 96%, at least about 97%, at least about 98%, at least about 99%, or more, sequence identity to SEQ ID NO: 3. In some embodiments, the repressible transactivator protein is a protein with the sequence of SEQ ID NO: 11. In some embodiments, the repressible transactivator protein is a protein comprising the sequence of SEQ ID NO: 11. In some embodiments, the repressible transactivator protein comprises an amino acid sequence having at least about 90%, at least about 95%, at least about 96%, at least about 97%, at least about 98%, at least about 99%, or more, sequence identity to SEQ ID NO: 11. A non-limiting example of VP16 is encoded by a nucleic acid sequence of SEQ ID NO: 4.

In some embodiments, the binding sequence for repressible transactivator protein comprises one or more operator sequences VanO. For example, the binding sequence can comprise two, more, three, four, five, six, seven, eight, nine, ten, or more VanO sequences. In some embodiments, the VanO sequence is the inverted repeat sequence ATTGGATCCAAT (SEQ ID NO: 9). In some embodiments, the binding sequence comprises one, two, three, four, five, six, seven, eight, nine, ten, fifteen, twenty, or more copies of SEQ ID NO: 9 or variants thereof. In some embodiments, the VanO sequence is a nucleic acid sequence having one, two, three, or four deletions, additions and/or substitutions in the nucleotide sequence relative to SEQ ID NO: 9. In some embodiments, the binding sequence comprises one, two, three, four, five, six, seven, eight, nine, ten, fifteen, twenty, or more copies of variant of SEQ ID NO: 9, where the variant of SEQ ID NO:9 is a nucleic acid sequence having one, two, three, or four deletions, additions and/or substitutions in the nucleotide sequence relative to SEQ ID NO: 9.

Various toxins can be used in the system, components and methods described herein. As would be appreciated by one of ordinary skill in the art, the type of the toxin is not particularly limited and a wide range of suitable gene products with varying toxicities can be selected by one of ordinary skill in the art according to the desired use. For example, the toxin can be any protein, or non-coding RNA, whose expression is sufficient to result in death of an insect, or dysfunction of a specific tissue (such as the flight muscle) required for the organism to have close to wildtype fitness in the wild. In some embodiments, gene products encoded by cell-signaling or cell-cycle genes can be used as the toxin. Examples of toxins include, but are not limited to, cell death proteins, restriction endonuclease, and microRNAs targeting essential genes. In some embodiments, the toxin is VP16 protein. In some embodiments, the VP16 protein is a protein having the sequence of SEQ ID NO: 12. In some embodiments, the VP16 protein is a protein comprising the sequence of SEQ ID NO: 12. In some embodiments, the VP16 protein comprises an amino acid sequence having at least about 90%, at least about 95%, at least about 96%, at least about 97%, at least about 98%, at least about 99%, or more, sequence identity to SEQ ID NO: 12. In some embodiments, the toxin is a pro-apoptotic protein that can induce cell death, such as proteins encoded by Drosophila hid, reaper, Nipp1Dam, and grim genes. The toxin is preferred to be expressed at levels sufficient to kill the organism, but with no or minimum environmental impact.

Various promoters can be used to be operably linked with the polynucleotide encoding the repressible transactivator protein in the regulatory genetic element. In some embodiments, the promoter can drive the expression of at least one of the repressible transactivator proteins in a host organism (e.g., a host insect) transformed with the vector containing the regulatory genetic element. The promoter can be naturally-occurring or non-naturally occurring. In some embodiment, the promoter that is operably linked with the polynucleotide encoding the repressible transactivator protein is a ubiquitous promoter. For example, the ubiquitous promoter can be Drosophila poly-ubiquitin promoter, baculovirus immediate early (iel) promoter, or any variant thereof. In some embodiments, the regulatory genetic element further comprises an enhancer for high expression of the repressible transactivator protein. For example, the enhancer can be baculovirus enhancer hr5 or any variants thereof. The nucleotide sequence of the promoter can also be modified, for example, for improving expression efficiency of the repressible transactivator protein. For example, the promoter can include one or more splice donors, one or more splice acceptors, and/or combination thereof. In some embodiments, the promoter includes a splice donor and a splice acceptor.

In some embodiments, the promoter is baculovirus immediate early (iel) promoter. In some embodiments, the promoter has a nucleic acid sequence of SEQ ID NO: 5. In some embodiments, the promoter comprises a nucleic acid sequence having at least about 90%, at least about 95%, at least about 96%, at least about 97%, at least about 98%, at least about 99%, or more, sequence identity to SEQ ID NO: 5. In some embodiments, the vector includes an enhancer having a nucleic acid sequence of SEQ ID NO: 6. In some embodiments, the enhancer comprises a nucleic acid sequence having at least about 90%, at least about 95%, at least about 96%, at least about 97%, at least about 98%, at least about 99%, or more, sequence identity to SEQ ID NO: 6.

The vectors can also comprise posttranscriptional regulatory elements, for example, to increase expression level of the repressible transactivator protein in a host cell. Non-limiting examples of the regulatory elements include transcription initiation regions and transcriptional termination regions. Examples of transcription termination region include, but are not limited to, polyadenylation signal sequences and associated downstream regions. In some embodiments, the transcriptional termination region is located downstream of the posttranscriptional regulatory element. In some embodiments, the transcriptional termination region is a polyadenylation signal sequence. In some embodiments, the transcriptional termination region is SV40 late poly(A) sequence.

In some embodiments, the vectors can include additional sequences that make the vectors suitable for replication and integration in eukaryotes. In some embodiments, the vectors disclosed herein can include a shuttle element that makes the vectors suitable for replication and integration in both prokaryotes and eukaryotes. In some embodiments, the vectors can include additional transcription and translation initiation sequences, such as promoters and enhancers; and additional transcription and translation terminators, such as polyadenylation signals.

In some embodiments, the vector comprises a VanDal repressible lethal system that includes a lethal genetic element and a regulatory element. The lethal genetic element and the regulatory element can be any of these elements disclosed herein. In the VanDal repressible lethal system, vanillic acid functions as an on/off switch for the expression of one or more toxins from the lethal genetic element. As shown in FIG. 1B, one or more VanO operator sequences can be located upstream of the P-hsp70^{min} promoter (described in e.g., Amin et al., Molecular and Cellular Biology, 7:1055-1062 (1987)) which drives the expression of a toxin gene. In the same system, the C-terminus of VanR is fused to transactivation domain VP16 to form a chimeric transcription factor VanR^{TF} (whose transcription is driven by a baculovirus immediate early (iel) promoter separated by a baculovirus enhancer (hr5), Bossin et al. Insect Mol. Biol. 16:37-47 (2007)). It is expected that in the absence of vanillic acid, the hr5iel expressed VanR^{TF} will bind to the VanO operator and activates a 3′-positioned minimal promoter P_hsp70min. This is expected to result in robust expression of the toxin, and ultimately death to the organism (see e.g., FIG. 1B). On the other hand, the addition of a small non-toxic dose (for example, about 50 uM or less) of vanillic acid to the insects diet is expected to trigger the release of VanR^{TF} from the VanO operator, resulting in the inactivation of P_hsp70^{min} promoter, loss of production of the toxin, and therefore survival of the organism (see e.g., FIG. 1C). Therefore, the Vanillic acid suppressible dominant lethal (VanDal) system described herein is expected to be able to control the size of insect populations.

The sequence of one non-limiting example of the vector disclosed herein is provided in SEQ ID NO: 1. In some embodiments, the vector carrying a VanDal repressible lethal system comprises a nucleotide sequence having at least about 75%, at least about 85%, at least about 80%, at least about 85%, at least about 90%, at least about 95%, at least about 96%, at least about 97%, at least about 98%, at least about 99%, or more, sequence identity to SEQ ID NO: 1.

In some embodiments, the vector comprises a feedback VanDal repressible lethal system that includes a lethal genetic element in which the expression of the one or more toxins and the repressible transactivator is driven from the same promoter. In such a VanDal repressible lethal system, vanillic acid also functions as an on/off switch for the expression of one or more toxins from the lethal genetic element. As shown in FIG. 3A, one or more VanO operator sequences can be located upstream of the P-hsp70^{min} promoter which drives the expression of VanR^{TF} (fusion protein in which the C-terminus of VanR is fused with VP16) and a toxin. In the same system, a T2A sequence is located between the coding region of VanR^{TF} and the toxin. It is expected that in the absence of vanillic acid, the leaky expression of the P-Hsp70^{min} promoter will produce small amount of VanR^{TF}-T2A-toxin, and separate VanR^{TF} and toxin proteins will be produced during translation by the self-cleaving t2a peptide. VanR^{TF} is expected to bind to the VanO operator, activates the P-hsp70^{min} promoter, and thus promote more expression of VanR^{FT} and the toxin. This is expected to result in robust expression of the toxin, and ultimately death to the organism. On the other hand, in the presence of vanillic acid, vanillic acid will prevent the small amount of the VanR^{TF} produced from the leaky expression from the P_hsp70^{min} promoter from binding to VanO sequences, and thus no additional toxin will be produced. The small amount of toxin produced from the leaky expression of the P_hsp70^{min} promoter is expected to be insufficient to kill or induce serious fitness costs to the organism. And thus, the organism can survive, and homozygous stocks of the organisms carrying the feedback VanDal repressible lethal system can be mass reared on vanillic acid supplemented diet. The feedback VanDal system can also be sex-specific. For example, as illustrated in FIG. 3B, a female-specific intron can be located in the coding region of VanR^{TF} to allow female-specific production of the VanR^{TF} protein. As other examples, a female-specific intron can be located in the coding region of the toxin to allow female-specific production of the toxin (FIG. 3C), and a female-specific intron can be located in each of the coding regions of the toxin and VanR^{TF} to allow female-specific production of the toxin and VanR^{TF} (FIG. 3D). The feedback VanDal system described herein is expected to be able to control the size of insect populations.

### Recombinant Insects

Also disclosed herein are recombinant insects comprising any of the lethal genetic elements disclosed herein. In some embodiments, the lethal genetic element comprises: a polynucleotide encoding one or more toxins, wherein the polynucleotide is operably linked with a first promoter; and a binding sequence for a repressible transactivator protein, wherein the binding sequence is operably linked with the first promoter and expression of the one or more toxins is activatable by the repressible transactivator protein; wherein the binding of the repressible transactivator protein to the binding sequence is repressible by vanillic acid, analogs or derivatives thereof. The recombinant insects can further comprise any of the regulatory genetic elements disclosed herein. In some embodiments, the regulatory genetic element comprises a polynucleotide encoding the repressible transactivator protein, wherein the polynucleotide is operably linked with a promoter.

As discussed above, the recombinant insects may be produced by transforming target insects with one or more vectors, such as plasmids, described herein. For example, the target insects can be transformed with a vector that includes both the lethal genetic element and the regulatory element, or the target insects can be co-transformed with a first vector the includes the lethal genetic element and a second vector that includes the regulatory element. In some embodiments, the lethal genetic element is integrated into the chromosomes of the target insects to produce the recombinant insects. In some embodiments, the recombinant insects have the regulatory genetic element integrated into their chromosomes as well.

The relative location of the lethal genetic element and the regulatory genetic element can vary. For example, the lethal genetic element and the regulatory genetic element can be located on the chromosome(s) of the recombinant insects. In some embodiments, the lethal genetic element and the regulatory genetic element are located on the same chromosome of the recombinant insects. For example, the lethal genetic element and the regulatory genetic element can be adjacent to each other on the same chromosome of the recombinant insects. For example, one or more insulators may be present between the adjacent lethal genetic element and the regulatory genetic element. In some embodiments, the lethal genetic element and the regulatory genetic element are located on different chromosome of the recombinant insects. In some embodiments, the lethal genetic element and the regulatory genetic element are configured to allow autonomous expression of the one or more toxins and the repressible transactivator protein in the recombinant insects. As another example, either lethal genetic element or the regulatory genetic element is present in the recombinant insects as an extrachromosomal DNA or part of an extrachromosomal DNA. In some embodiments, the lethal genetic element is located in the chromosome of the recombinant insects and the regulatory element is part of an extrachromosomal DNA (e.g., plasmid DNA, or mitochondrial DNA) in the recombinant insects.

The methods, systems and components described herein are applicable to various types of insects. For example, the insect can be a direct pest or indirect pest. As used herein, “direct pests” refers to insects that can cause damage at one or more stage of their life cycle by, for example, eating crops or damaging animals. The New World screw-worm fly Cochliomyia hominivorax, for example, is a direct pest of cattle, and the spotted wing Drosophila, Drosophila suzukii is pest of many fruit crops. As used herein, “indirect pests” refers to insects that are vectors of human diseases, such as mosquitoes which carry malaria. Indirect pests of organisms other than humans, such as livestock or plants are also known. Examples of insects include, but are not limited to, Asian citrus psyllid (diaphorini citrii), Australian sheep blowfly (Lucilia cuprina, Asian tiger mosquito (Aedes albopictus); Japanese beetle (Popilla japonica), White-fringed beetle (Graphognatus spp.), Citrus blackfly (Aleurocanthus woglumi), Oriental fruit fly (Dacus dorsalis), Olive fruit fly (Dacus oleae), tropical fruit fly (Dacus cucurbitae, Dacus zonatus), Mediterranean fruit fly (Ceratitis capitata), Natal fruit fly (Ceratitis rosa), Chemy fruit fly (Rhagoletis cerasi), Queensland fruit fly (Bactrocera tryoni), Caribbean fruit fly (Anastrepha suspensa), imported fire ants (Solenopis richteri, Solenopis invicta), Gypsy moth (Lymantria dispar), Codling moth (Cydia pomonella), Brown tail moth (Euproctis chrysorrhoea), yellow fever mosquito (Aedes aegypti), malaria mosquitoes (Anopheles gambiae, Anopheles stephansi), New world screwworm (Cochliomyia hominivorax), Old World Screwworm (Chrysomya bezziana), Tsetse fly (Glossina spp), Boll weevil (Anthonomous grandis), Damsel fly (Enallagma hageni), Dragonfly (Libellula luctuosa), and rice stem borer (Tryporyza incertulas). In some embodiments, the insects are vectors of human disease or agriculture pest. In some embodiments, the insects are wild insects. In some embodiments, the insects are mosquitoes or flies (for example fruit flies). The mosquitoes can be, for example, Aedes sp. or Anopheles sp. In some embodiments, the mosquito is yellow fever mosquito (Aedes aegypti), malaria mosquitoes (Anopheles gambiae, Anopheles stephansi), and Asian tiger mosquito (Aedes albopictus).

The lethal genetic element and/or the regulatory genetic element described herein can be sex-specific in terms of expression of the one or more toxins and/or the repressible transactivator protein. For example, a female specific intron can be encoded within the polynucleotide encoding the repressible transactivator protein such that a nonfunctional repressible transactivator protein can be produced through improper splicing when expressed in male insects, and functional repressible transactivator protein can be produced through proper splicing when expressed in male insects. As another example, a female specific intron can be encoded within the polynucleotide encoding the one or more toxins such that nonfunctional toxin(s) can be produced through improper splicing when expressed in male insects, and functional toxin(s) can be produced through proper splicing when expressed in male insects. In some embodiments, a first female intron is encoded within the polynucleotide encoding the repressible transactivator protein and a second female intron is encoded within the polynucleotide encoding the one or more toxins. In another non-limiting example, a female specific promoter can be used to drive expression of the repressible transactivator protein so that the repressible transactivator protein is only expressed in female insects and not in male insects. Non-limiting examples of the female specific intron include Drosophila tra intron, mosquito dsx intron, and any variants thereof.

FIGS. 2A-D show a number of non-limiting embodiments of the female-specific VanDal repressible lethal system. In the female-specific VanDal repressible lethal system shown in FIG. 2A, a VanO operator sequence is located upstream of the P-hsp70^{min} promoter, which drives the expression of a toxin gene. In the same system, the C-terminus of VanR is fused to transactivation domain VP16 to form a chimeric transcription factor VanR^{TF} (whose transcription is driven by a baculovirus immediate early (iel) promoter separated by a baculovirus enhancer (hr5)), and a female specific intron is located within the polynucleotide encoding VanR^{TF} to enable female-specific splicing. The female-specific VanDal repressible lethal system shown in FIG. 2B has a similar design to that of the VanDal system shown in FIG. 2A except that the female-specific intron is located within the polynucleotide encoding the toxin. The female-specific VanDal repressible lethal system shown in FIG. 2C includes two female specific introns—one located within the polynucleotide encoding the toxin and one located within the polynucleotide encoding VanR^{TF}. In the female-specific VanDal repressible lethal system shown in FIG. 2D, the expression of VanR^{TF} is driven by a female-specific promoter.

In some embodiments, the recombinant insect comprises a feedback VanDal repressible lethal system that includes a lethal genetic element in which the expression of the one or more toxins and the repressible transactivator is driven from the same promoter. In such a VanDal repressible lethal system, vanillic acid also functions as an on/off switch for the expression of one or more toxins from the lethal genetic element. In some embodiments, the recombinant insect comprises a lethal genetic element that comprises: a polynucleotide comprising a first coding region for a toxin, a second coding region for a repressible transactivator protein, and a 2A sequence located between the first coding region and the second coding region, wherein the polynucleotide is operably linked with a promoter; and a binding sequence for the repressible transactivator protein, wherein the binding sequence is operably linked with the promoter and expression of the toxin is activatable by the repressible transactivator protein; wherein the binding of the repressible transactivator protein to the binding sequence is repressible by vanillic acid, analogs or derivatives thereof. In some embodiments, the first coding region, the second coding region, or both the first and the second coding region further comprise a female specific intron. In some embodiments, the second coding region is located upstream of the first coding region. As illustrated in FIG. 3A, a single promoter (e.g, P-hsp70^{min} promoter) which is downstream of one or more VanO operator sequences can be used to drive the expression of a repressible transactivator protein (e.g., VanR^{TF}) and one or more toxins, and a 2A sequence (e.g., a T2A sequence) can be located between the coding region of VanR^{TF} and the toxin to allow production of separate VanR^{TF} and toxin proteins. It is expected that in the absence of vanillic acid, the leaky expression of the P-Hsp70^{min} promoter will produce small amount of VanR^{TF} and toxin proteins. VanR^{TF} is expected to bind to the VanO operator, activates the P-hsp70^{min} promoter, and thus result in robust expression of VanR^{FT} and the toxin, and ultimately death in the recombinant insects. On the other hand, in the presence of vanillic acid, vanillic acid will prevent the small amount of the VanR^{TF} produced from the leaky expression from the P_hsp70^{min} promoter from binding to VanO sequences, and thus no additional toxin will be produced. The small amount of toxin produced from the leaky expression of the P_hsp70^{min} promoter is expected to be insufficient to kill or induced serious fitness costs to the organism. And thus, the recombinant insect can survive, and homozygous stocks of the organisms carrying the feedback VanDal repressible lethal system can be mass reared on vanillic acid supplemented diet.

The feedback VanDal system as described herein can be sex-specific. For example, as illustrated in FIG. 3B, a female-specific intron can be located in the coding region of VanR^{TF} to allow female-specific production of the VanR^{TF} protein. As other examples, a female-specific intron can be located in the coding region of the toxin to allow female-specific production of the toxin (FIG. 3C), and a female-specific intron can be located in each of the coding regions of the toxin and VanR^{TF} to allow female-specific production of the toxin and VanR^{TF} (FIG. 3D). The feedback VanDal system described herein is expected to be able to control the size of insect populations.

### Methods for Regulating Insect Populations

Also disclosed herein are methods for regulating insect populations. For example, the methods can be used to control, suppress, or eliminate one or more insect populations. In some embodiments, the methods include breeding a stock of male and female organisms under permissive conditions (e.g., under a vanillic acid supplemented diet), allowing the survival of males and females to produce a dual sex biological control agent; and distributing the dual sex biological control agent into an environment that needs biological control of the organisms. As disclosed herein, the expression of the lethal genetic elements can be controlled under permissive conditions in a laboratory, factory or other regulated system, for example, to allow growth of a normal population, e.g. insect stock with both sexes, in the presence of vanillic acid. In some embodiments, prior to release of the factory or laboratory stock into the environment the conditions can be manipulated to ensure only single sex populations of the organism are distributed into the environment.

In some embodiments, the methods include: providing a stock of recombinant insects, wherein at least one of the recombinant insect comprises a lethal genetic element, wherein the lethal genetic element comprises: a first polynucleotide encoding one or more toxins, wherein the first polynucleotide is operably linked with a first promoter; and a binding sequence for a repressible transactivator protein, wherein the binding sequence is operably linked with the first promoter and expression of the one or more toxins is activatable by the repressible transactivator protein; wherein the binding of the repressible transactivator protein to the binding sequence is repressible by vanillic acid, analogs or derivatives thereof; and distributing the stock of recombinant insects into an environment lacking vanillic acid, analogs or derivatives thereof, whereby individual recombinant insects breed with insects in the environment to produce offspring expressing the one or more toxins. In some embodiments, the method further includes breeding the stock of recombinant insects in the presence of vanillic acid, analogs or derivatives thereof before distributing the stock of recombinant insects into the environment.

The methods disclosed herein can also be used to provide sex-specific regulation of insect populations. As disclosed above, the expression of the lethal genetic element can sex-specific. In some embodiments, the expression of the one or more toxins is sex-specific. In some embodiments, the expression of the one or more toxins is controlled by a sex-specific promoter, a sex-specific enhancer, or by sex-specific splicing. In some embodiments, the expression of the lethal genetic element can be female specific. For example, the expression of the one or more toxins on the lethal genetic element can be female specific. As another example, the expression of the repressible transactivator can be female specific. In some embodiments, both the expression of the one or more toxins and the expression of the repressible tranactivator are female specific. In some embodiments, the methods can be used to control, suppress, or eliminate one or more female insect populations. In some embodiments, only female recombinant insects are distributed to the environment. In a non-limiting example of the methods, in the presence of vanillic acid supplemented diet, the lethal genetic element in the recombinant insects does not express toxin, and a stock of the recombinant insects can be bred. Imposition of restrictive conditions, that is depriving the insect diet of vanillic acid, allows one sex (for example, females) to be killed. The remaining sex (males) can be released to the environment, and the lethal genetic element can be passed on to at least some offspring resulting from any sexual reproduction between the males and a wild-type insect of the same species. The conditional dominant lethal genetic system is selected such that expression of the lethal system occurs in the natural environment. As a result, for a female-specific lethal genetic element, all females which result from the mating are then killed or rendered non-viable due to the action of the lethal genetic element and regulatory element, while the males survive to pass on the genetic elements to the next generation in a proportion of cases. As a result, biological control can be achieved.

### EXAMPLE

Additional embodiments are disclosed in further detail in the following examples, which are not in any way intended to limit the scope of the claims.

### Example 1

### Construction of VanDal System

To engineer a dominant vanillic acid-repressible lethal system (VanDal system), a plasmid was constructed, in which the C-terminus of VanR was fused with the Herpes simplex virus transactivation domain VP16, generating a chimeric transcription factor (vanRTF) (as descried in Hagmann et al., 1997). The chimeric transcription factor VanR^{TF} was driven at high levels, in a ubiquitous pattern, using a baculovirus immediate early (iel) promoter separated by a baculovirus enhancer (hr5) (as described in Bossin et al., 2007). On the same plasmid, but facing in the opposite direction, VanO operator sequences were placed upstream of the P_hsp70^{min} promoter (as described in Amin et al., 1987), and the P_hsp70^{min} promoter drove the expression of toxin VP16. The vanillic acid-repressible lethal system resides in a single piggyback transposable element that also includes a positive selection marker gene, (Opie2-dsRed), to facilitate the identification of transgenic insects.

It is expected that in the absence of vanillic acid, the hr5iel expressed VanRTF will bind to the VanO operator sequences and activates a 3′-positioned minimal promoter, P_hsp70^{min}. This is expected to result in robust expression of the toxin, and ultimately death to the organism (see e.g., FIG. 1B). On the other hand, the addition of a small non-toxic dose (for example, about 50 uM or less) of vanillic acid to the insects diet is expected to trigger the release of VanR^{TF} from the VanO operator, resulting in the inactivation of P_hsp70min promoter, loss of production of the toxin, and therefore survival of the organism (see e.g., FIG. 1C). Therefore, the Vanillic acid suppressible dominant lethal (VanDal) system described herein is expected to be able to control the size of insect populations.

### Example 2

### Evaluation of VanDal System in Biological Control

In this example, in vivo functionality of the VanDal system prepared according to the general procedure of Example 1 was evaluated in Drosophila melanogater, an insect model organism. Healthy transgenic lines that only survive when fed a diet supplemented with a low dose vanillic acid were established. To simulate a release into the wild, two crosses in the absence of vanillic acid were set up: 1) homozygous VanDal males mated separately to wildtype virgin females and 2) homozygous Vandal females mated separately to wildype males. In both cases no surviving progeny were produced, resulting in a collapse of the population as expected.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A recombinant insect, comprising a lethal genetic element that comprises:
a first polynucleotide encoding one or more toxins, wherein the first polynucleotide is operably linked with a first promoter; and
a binding sequence for a repressible transactivator protein, wherein the binding sequence is operably linked with the first promoter and expression of the one or more toxins is activatable by the repressible transactivator protein;
wherein the binding of the repressible transactivator protein to the binding sequence is repressible by vanillic acid, analogs or derivatives thereof.

2. The recombinant insect of claim 1, further comprising a regulatory genetic element that comprises a second polynucleotide encoding the repressible transactivator protein, wherein the second polynucleotide is operably linked with a second promoter.

3. The recombinant insect of claim 2, wherein the lethal genetic element and the regulatory element are located on the same chromosome of the recombinant insect.

4. The recombinant insect of claim 2, wherein the lethal genetic element and the regulatory element are located on different chromosomes of the recombinant insect.

5. The recombinant insect of claim 1, wherein the repressible transactivator protein is VanR.

6. The recombinant insect of claim 1, wherein the binding sequence for the repressible transactivator protein is VanO operator sequence.

7. The recombinant insect of claim 1, wherein at least one of the one or more toxins is a cell death protein, a restriction endonuclease, or a microRNA.

8. The recombinant insect of claim 1, wherein the first promoter is a P_hsp70^{min} promoter, a CMV^{min} promoter, a drosophila minimal P-element promoter, or any variant thereof.

9. The recombinant insect of claim 1, wherein the first promoter is a female specific promoter.

10. The recombinant insect of claim 2, wherein the second promoter is a ubiquitous promoter.

11. The recombinant insect of claim 10, wherein the ubiquitous promoter is selected from the group consisting of baculovirus immediate early promoter.

12. The recombinant insect of claim 1, wherein the first polynucleotide encoding one or more toxins, the second polynucleotide encoding the repressible transactivator protein, or both polynucleotides comprise a female specific intron.

13. A vector, comprising a lethal genetic element that comprises:
a first polynucleotide encoding one or more toxins, wherein the first polynucleotide is operably linked with a first promoter; and
a binding sequence for a repressible transactivator protein, wherein the binding sequence is operably linked with the first promoter and expression of the one or more toxins is activatable by the repressible transactivator protein;
wherein the binding of the repressible transactivator protein to the binding sequence is repressible by vanillic acid, analogs or derivatives thereof

14. The vector of claim 13, further comprising a regulatory genetic element that comprises a second polynucleotide encoding the repressible transactivator protein, wherein the second polynucleotide is operably linked with a second promoter.

15. The vector of claim 13, wherein the lethal genetic element and the regulatory genetic element are configured to allow autonomous expression of the one or more toxins and the repressible transactivator protein.

16. The vector of claim 15, wherein the lethal genetic element and the regulatory genetic element are separated by one or more insulators.

17. A method for regulating insect population, comprising:
providing a stock of recombinant insects, wherein at least one of the recombinant insect comprises a lethal genetic element, wherein the lethal genetic element comprises:
a first polynucleotide encoding one or more toxins, wherein the first polynucleotide is operably linked with a first promoter; and
a binding sequence for a repressible transactivator protein, wherein the binding sequence is operably linked with the first promoter and expression of the one or more toxins is activatable by the repressible transactivator protein;
wherein the binding of the repressible transactivator protein to the binding sequence is repressible by vanillic acid, analogs or derivatives thereof; and
distributing the stock of recombinant insects into an environment lacking vanillic acid, analogs or derivatives thereof, whereby individual recombinant insects breed with insects in the environment to produce offspring expressing the one or more toxins.

18. The method of claim 17, wherein the first polynucleotide further comprises a coding region for the repressible transactivator protein, wherein a 2A sequence is located between the coding region for the repressible transactivator protein and the coding region of the one or more toxins.

19. The method of claim 17, wherein breeding the stock of recombinant insects in the presence of vanillic acid, analogs or derivatives thereof before distributing the stock of recombinant insects into the environment.

20. The method of claim 17, wherein the expression of the one or more toxins is sex-specific.

21. The method of claim 20, wherein the expression of the one or more toxins is controlled by a sex-specific promoter, a sex-specific enhancer, or by sex-specific splicing.

22. The method of claim 20, wherein only male recombinant insects are distributed to the environment.

23. A recombinant insect, comprising a lethal genetic element that comprises:
a polynucleotide comprising a first coding region for a toxin, a second coding region for a repressible transactivator protein, and a 2A sequence located between the first coding region and the second coding region, wherein the polynucleotide is operably linked with a promoter; and
a binding sequence for the repressible transactivator protein, wherein the binding sequence is operably linked with the promoter and expression of the toxin is activatable by the repressible transactivator protein;
wherein the binding of the repressible transactivator protein to the binding sequence is repressible by vanillic acid, analogs or derivatives thereof.

24. The recombinant insect of claim 23, wherein the first coding region, the second coding region, or both the first and the second coding region comprise a female specific intron.

25. The recombinant insect of claim 23, wherein the second coding region is located upstream of the first coding region.


SEQUENCE LISTING (12 sequences)

SEQ ID NO: 1 - DNA, 11167 bp, Artificial Sequence
  plasmid
accgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacgctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatccgagatcggccggcctagaagctttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctagagtcctacaggaacaggtggtggcggccctcggtgcgctcgtactgctccacgatggtgtagtcctcgttgtgggaggtgatgtccagcttggagtccacgtagtagtagccgggcagctgcacgggcttcttggccatgtagatggacttgaactccaccaggtagtggccgccgtccttcagcttcagggccttgtggatctcgcccttcagcacgccgtcgcgggggtacaggcgctcggtggaggcctcccagcccatggtcttcttctgcattacggggccgtcggaggggaagttcacgccgatgaacttcaccttgtagatgaagcagccgtcctgcagggaggagtcctgggtcacggtcaccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactccttgatgacgttcttggaggagcgcaccatcaccagagacaggttgcggcggcggttggatggcgtgggcgcgttggcgttgttggaccggctcatgttgtgtcgctgtaacagatgctgttcaactgtgtttaccagatcgttgcgggctgtatttataggcgcgataagcgggacgggcgcctcgtgtccggtcacgcgcatgagataacgcgcggctgatatggaggcgcgtcctgttccgataaggagttgcgtccggctgcggttagcaacacaggaagctggcgtcctgtcacgataagacaacactcgtccggtccgataatgtgattcgtacgtgacaggacgcgacccgataaggccggcctacgtgactgccgacacgtacttttttgcactgcaaaaaggttcaatgtgtggtagtgtatttggagcgtatacaacggtgtagactatttatgtaaaatagtctacgaaacgtagagtttgtactatgtatgggcccgcgtgcaaaagcgtgtttttttgcagtgcaaaaaagttggtggtggggaggccaccgagtatggtttaaactggaaccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattccattcattttatgtttcaggttcagggggaggtgtgggaggttttttaaagcaagtaaaacctctacaaatgtggtatggctgattatgatctctagtcaaggcactatacatcaaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatcctcgagctacccgccatactcatcgatgcccagcgcgtcggtgaacatttgctcgaactcgaagtcggccatgtccagggcgccgtacggggcgctatcgtggggcgtgaagcccggtcccgggctatctccatcgcccagcatatccaggtcgaaatcgtccagggcgtcggcgtgggccattgccacatcctctccatccaggtgcagctcgtcgcccaggctcacatcggtcggcggggcggtgctcaggcggcgcgtgtgtccggcgggcaggaagctcaggcggggggcggccaggccggcttcctccggggcatcgtcatccggcaggtccagcagtccctcgatggtgctgccatagttgttcttggtacgggcgcggctgtaggcgctgccgtcggcgcgaatgctccacgccgcgcccagcggcgcgccggcgctcgccgcggcctcgaagaccttggcgttacgaatggcggccagggcatggtcgcgcatgatccgctcggccccctcggcgtcgccgcacgagaccgcgtccagcaccgcctgatgctggcgatgggcggccagaaggtgttcgtactcggccggcaagtccatcagatccagggccagcgcaccggccgccgcgaacggctcgaaaccattgcgcgccagggcgctctcgacagcgccgttgcccgcagcgctgaccagcgtgtcatggaaggcctgattgtacgcggcgtagcgatccaggtcctcgccgttaagccgcccagccgcgaacagcgcttcgccctcggcgatcaaggcgacgaaccgcgcgtgggtctccgccgtcattccgcgctcggccagacgccgggccgcaaagccctccagcacccctcgcacctcgatggcgtcgcggatctggtcgctggacacgcctcgggcggcataaccccgcgcgcccagccgcaccacgagcccttcctgctccagactccgcaaggccgtgcgcacgggcatgcgcgagacgcccagcgccgccgctgtcgggatttcggcgatacgctcgccgctcttgatctcgcccgaggcgatcatcttgcgaagagccatcatcacgcgctggcccggctttatgcgcggcatgtccatgcggccgcgtcacttggttgttcacgatcttgtcgccgccagtgtcaacttgcaactgaaacaatatccaacatgaacgtcaatttatactgccctaatggcgaacacgataacaatatttcttttattatgccctctaaaaccaacgcggttatcgtttatttattcaaattagatatagaacatccgccgacatacaatgttaatgcaaaaacgcgtttggtgagcggatacgaaaacagtcggccgataaacattaatctgaggtcgataacaccgtccttgaacggaacacgaggagcgtacgtgatcagctgcattcgcgcgccgcgcctttatcgagatttatttgcatacaacaagtacactgcgccgttgggatttgtggtaacgcgcacacatgcagagctgcaagtgtggcacattttgtctgtgcgcaaaacctttgaagccaaaagtacgaggtccgttacgggcatgctagcgcacacggacaatggacccgacaaattctacgccaaggatttaatgataatgtcgggcaacgtatccgttcattttatcaataacctacaaaaatgtcgcgcgcatcacaaagacatcgacgcgcgtagaattctacccgtaaagcgagtttagttatgagccatgtgcaaaacatgacatcagcttttatttttataacaaatgacatcatttcttgattgtgttttacacgtagaattctactcgtaaagcgagttcagttttgaaaaacaaatgacatcatctttttgattgtgctttacaagtagaattctacccgtaaatcaagttcggttttgaaaaacaaatgagtcatattgtatgatatcatattgcaaaacaaatgactcatcaatcgatcgtgcgttacacgtagaattctactcgtaaagcgagtttatgagccgtgtgcaaaacatgacatcatctcgatttgaaaaacaaatgacatcatccactgatcgtgcgttacaagtagaattctactcgtaaagccagttcggttatgagccgtgtgcaaaacatgacatcagcttatgactcatacttgattgtgttttacgcgagatctcaatatttaaatggcgggggccgcgattacgatcaagattggatccaatcttgacgtccgtttgattacgatcaagattggatccaatcttgacgtccgtttgaccatggcgattacgatcaagattggatccaatcttgacgtccgtttgattacgatcaagattggatccaatcttgacgtccgtttgattacgatcaagattggatccaatcttgacgtccgtttttaattaaattggatccaatagcgctattggatccaatagcgccggagtataaatagaggcgcttcgtctacggagcgacaattcaattcaaacaagcaaagtgaacacgtcgctaagcgaaagctaagcaaataaacaagcgcagctgaacaagctaaacaatctgccctaggatgggcagcgcctacagccgcgcccgtaccaagaacaactatggcagcaccatcgagggactgctggacctgccggatgacgatgccccggaggaagccggcctggccgccccccgcctgagcttcctgcccgccggacacacgcgccgcctgagcaccgccccgccgaccgatgtgagcctgggcgacgagctgcacctggatggagaggatgtggcaatggcccacgccgacgccctggacgatttcgacctggatatgctgggcgatggagatagcccgggaccgggcttcacgccccacgatagcgccccgtacggcgccctggacatggccgacttcgagttcgagcaaatgttcaccgacgcgctgggcatcgatgagtatggcgggtaggcgatcgctggaaccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattccattcattttatgtttcaggttcagggggaggtgtgggaggttttttaaagcaagtaaaacctctacaaatgtggtatggctgattatgatctctagtcaaggcactatacatcaaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccttaagcctaaaccccgggcgagctcgaattaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggttcaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgtatatctcaacgctgtctgtgagattgtcgtattctagcctttttagtttttcgctcatcgacttgatattgtccgacacattttcgtcgatttgcgttttgatcaaagacttgagcagagacacgttaatcaactgttcaaattgatccatattaacgatatcaacccgatgcgtatatggtgcgtaaaatatattttttaaccctcttatactttgcactctgcgttaatacgcgttcgtgtacagacgtaatcatgttttcttttttggataaaactcctactgagtttgacctcatattagaccctcacaagttgcaaaacgtggcattttttaccaatgaagaatttaaagttattttaaaaaatttcatcacagatttaaagaagaaccaaaaattaaattatttaatcgaccagttaatcaacgtgtacacagagcgcaaaaaacacgcagcccgacgtgttggctaaaattattaaatcaacttgtgttatagtcacgatttgccgtccaacgtgttcctcaaaaagttgaagaccaacaagtttacggacactagttaattatttgattttgccccacttcattttgtgggatcacaattttgttatattttaaacaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggata

SEQ ID NO: 2 - DNA, 732 bp, Artificial Sequence
  VanR
atggacatgccgcgcataaagccgggccagcgcgtgatgatggctcttcgcaagatgatcgcctcgggcgagatcaagagcggcgagcgtatcgccgaaatcccgacagcggcggcgctgggcgtctcgcgcatgcccgtgcgcacggccttgcggagtctggagcaggaagggctcgtggtgcggctgggcgcgcggggttatgccgcccgaggcgtgtccagcgaccagatccgcgacgccatcgaggtgcgaggggtgctggagggctttgcggcccggcgtctggccgagcgcggaatgacggcggagacccacgcgcggttcgtcgccttgatcgccgagggcgaagcgctgttcgcggctgggcggcttaacggcgaggacctggatcgctacgccgcgtacaatcaggccttccatgacacgctggtcagcgctgcgggcaacggcgctgtcgagagcgccctggcgcgcaatggtttcgagccgttcgcggcggccggtgcgctggccctggatctgatggacttgccggccgagtacgaacaccttctggccgcccatcgccagcatcaggcggtgctggacgcggtctcgtgcggcgacgccgagggggccgagcggatcatgcgcgaccatgccctggccgccattcgtaacgccaaggtcttcgaggccgcggcgagcgccggcgcgccgctgggcgcggcgtggagcattcgcgccgac

SEQ ID NO: 3 - DNA, 1125 bp, Artificial Sequence
  VanRTF
atggacatgccgcgcataaagccgggccagcgcgtgatgatggctcttcgcaagatgatcgcctcgggcgagatcaagagcggcgagcgtatcgccgaaatcccgacagcggcggcgctgggcgtctcgcgcatgcccgtgcgcacggccttgcggagtctggagcaggaagggctcgtggtgcggctgggcgcgcggggttatgccgcccgaggcgtgtccagcgaccagatccgcgacgccatcgaggtgcgaggggtgctggagggctttgcggcccggcgtctggccgagcgcggaatgacggcggagacccacgcgcggttcgtcgccttgatcgccgagggcgaagcgctgttcgcggctgggcggcttaacggcgaggacctggatcgctacgccgcgtacaatcaggccttccatgacacgctggtcagcgctgcgggcaacggcgctgtcgagagcgccctggcgcgcaatggtttcgagccgttcgcggcggccggtgcgctggccctggatctgatggacttgccggccgagtacgaacaccttctggccgcccatcgccagcatcaggcggtgctggacgcggtctcgtgcggcgacgccgagggggccgagcggatcatgcgcgaccatgccctggccgccattcgtaacgccaaggtcttcgaggccgcggcgagcgccggcgcgccgctgggcgcggcgtggagcattcgcgccgacggcagcgcctacagccgcgcccgtaccaagaacaactatggcagcaccatcgagggactgctggacctgccggatgacgatgccccggaggaagccggcctggccgccccccgcctgagcttcctgcccgccggacacacgcgccgcctgagcaccgccccgccgaccgatgtgagcctgggcgacgagctgcacctggatggagaggatgtggcaatggcccacgccgacgccctggacgatttcgacctggatatgctgggcgatggagatagcccgggaccgggcttcacgccccacgatagcgccccgtacggcgccctggacatggccgacttcgagttcgagcaaatgttcaccgacgcgctgggcatcgatgagtatggcgggtag

SEQ ID NO: 4 - DNA, 396 bp, Artificial Sequence
  VP16
atgggcagcgcctacagccgcgcccgtaccaagaacaactatggcagcaccatcgagggactgctggacctgccggatgacgatgccccggaggaagccggcctggccgccccccgcctgagcttcctgcccgccggacacacgcgccgcctgagcaccgccccgccgaccgatgtgagcctgggcgacgagctgcacctggatggagaggatgtggcaatggcccacgccgacgccctggacgatttcgacctggatatgctgggcgatggagatagcccgggaccgggcttcacgccccacgatagcgccccgtacggcgccctggacatggccgacttcgagttcgagcaaatgttcaccgacgcgctgggcatcgatgagtatggcgggtag

SEQ ID NO: 5 - DNA, 600 bp, Artificial Sequence
  iel promoter
gcgtcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagatcgtgaacaaccaagtgac

SEQ ID NO: 6 - DNA, 482 bp, Artificial Sequence
  hr5 enhancer
cgcgtaaaacacaatcaagtatgagtcataagctgatgtcatgttttgcacacggctcataaccgaactggctttacgagtagaattctacttgtaacgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgc

SEQ ID NO: 7 - DNA, 130 bp, Artificial Sequence
  P-hsp70min promoter
agcgccggagtataaatagaggcgcttcgtctacggagcgacaattcaattcaaacaagcaaagtgaacacgtcgctaagcgaaagctaagcaaataaacaagcgcagctgaacaagctaaacaatctgc

SEQ ID NO: 8 - DNA, 248 bp, Artificial Sequence
  tra intron
gtacccactatatagtataaccgatctgatcagattctgatccagtgccatctttttgttgttttttttctagtgtcatattgtgtgaaatgtgaaatggatgagaatgaacgctggacgaccagacagagaaagagaagctaggacaataggactctcaactgcgcattacgtggattccgtctccgacgatgcgccaaacactatgcgttagatgcattgactaaccgcacactcttttcacatag

SEQ ID NO: 9 - DNA, 12 bp, Artificial Sequence
  VanO
attggatccaat

SEQ ID NO: 10 - protein, 244 aa, Artificial Sequence
  VanR
MDMPRIKPGQRVMMALRKMIASGEIKSGERIAEIPTAAALGVSRMPVRTALRSLEQEGLVVRLGARGYAARGVSSDQIRDAIEVRGVLEGFAARRLAERGMTAETHARFVALIAEGEALFAAGRLNGEDLDRYAAYNQAFHDTLVSAAGNGAVESALARNGFEPFAAAGALALDLMDLPAEYEHLLAAHRQHQAVLDAVSCGDAEGAERIMRDHALAAIRNAKVFEAAASAGAPLGAAWSIRAD

SEQ ID NO: 11 - protein, 374 aa, Artificial Sequence
  VanR-TF
MDMPRIKPGQRVMMALRKMIASGEIKSGERIAEIPTAAALGVSRMPVRTALRSLEQEGLVVRLGARGYAARGVSSDQIRDAIEVRGVLEGFAARRLAERGMTAETHARFVALIAEGEALFAAGRLNGEDLDRYAAYNQAFHDTLVSAAGNGAVESALARNGFEPFAAAGALALDLMDLPAEYEHLLAAHRQHQAVLDAVSCGDAEGAERIMRDHALAAIRNAKVFEAAASAGAPLGAAWSIRADGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 12 - protein, 131 aa, Artificial Sequence
  VP16
MGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG